(19) United States
(12) Patent Application Publication
(10) Pub. No.: US 2008/0221599 A1
(43) Pub. Date: Sep. 11, 2008

(54) Devices, methods, and kits for gastrointestinal procedures

(76) Inventor: Niel F. Starksen, Los Altos Hills, CA (US)

Correspondence Address: MORRISON & FOERSTER LLP, 755 PAGE MILL RD, PALO ALTO, CA, 94304-1018, US

(21) Appl. No.: 11/715,129

(22) Filed: Mar. 6, 2007

(51) Int. Cl. A61F 2/00

(52) U.S. Cl. 606/157

(57) ABSTRACT

Devices, methods and kits for restricting a portion of a GI tract by tightening tissue are provided. The devices, methods and kits can be used to treat GERD or obesity. The devices, methods, and kits do not require the formation of plications in tissue walls and may result in faster application, reduced surgical trauma, reduced risk, and reduced cost. The devices comprise a plurality of tissue-engageable anchors coupled to a tether. Each anchor is secured to a tissue wall of the GI tract. The anchors are secured to the tissue wall in a manner that may minimize tissue damage. The tether is configured to be cinched to draw the anchors together, which in turn draws the tissue secured to the anchors together, thereby tightening tissue to restrict a portion of the GI tract.

### FIELD

The devices, methods and kits described here generally relate to the field of gastrointestinal (GI) surgery. In particular, the devices, methods and kits relate to restriction of tissue in a GI tract. The devices, methods and kits can be used to treat obesity, gastroesophageal reflux disease (GERD), and the like.

### BACKGROUND

Morbid obesity represents a serious and rapidly growing health problem. Complications of obesity are numerous and include cardiac failure, diabetes, and hypertension. Thus, the high rate of obesity increases health care costs and can lead to lowered life expectancies. A variety of surgical and non-surgical techniques have been developed to treat morbid obesity. Conventional bariatric surgical techniques have followed two general approaches. First, malabsorptive or gastric bypass techniques have been developed to alter the GI tract to reduce absorption of caloric content of ingested food. Gastric bypass procedures involve bypassing a portion of the small intestine to reduce absorption of nutrients. Second, gastric restriction techniques have been developed to reduce the volume of a part of the GI system (e.g., the stomach) to prevent ingestion of large quantities of food. For example, “stomach stapling” techniques can be used to reduce the volume of a stomach. Alternatively, an adjustable gastric band can be applied around the outside of a stomach to restrict part of the stomach. Both of these techniques generally require open surgery or laparoscopic surgery and can have high rates of complication.

In GERD, weakness or defect in the gastroesophageal junction region between the stomach and the esophagus leads to backflow of stomach acid and contents into the esophagus. One conventional surgical procedure to treat GERD is called a fundoplication and involves the use of staples or sutures to fold the upper part of the stomach around the lower esophageal sphincter to strengthen the sphincter muscle to prevent reflux. Such surgical procedures typically involve access through the abdomen or thoracic region. Furthermore, they may result in complications such as difficulty in swallowing, or vomiting. In addition, staples or sutures may loosen over time, leading to recurrence of the condition. One FDA-approved non-surgical endoscopic procedure for treatment of GERD involves the insertion of a plicator transesophageally to make a fold in tissue in the gastroesophageal junction and secure the fold to strengthen the junction.

GI restriction techniques that require the formation of multiple individual plications in a tissue wall can be very time consuming, subjecting the patient to extended or multiple surgeries and hence to higher surgical risk. Further, devices that secure tissue plications must be adjusted carefully to avoid excess tissue compression that can lead to tissue necrosis.

Accordingly, improved devices, methods and kits for performing GI procedures for treating obesity and GERD are needed. For example, devices, method and kits for restricting a GI tract that do not require formation of individual plications may reduce procedure time, costs, and/or risks. Devices, methods and kits that can be used intraluminally to minimize trauma and complications are desired.

### SUMMARY

Devices, methods and kits for treating obesity and GERD through tightening of tissue are provided. The devices, methods and kits can enable simplified GI procedures that may minimize the time and extent of equipment required to perform them. The devices, methods and kits can be used intraluminally to reduce trauma associated with the GI procedures.

The devices restrict a portion of a GI tract for treating obesity or GERD. The devices comprise a plurality of tissue-engageable anchors coupled to a tether. Each anchor comprises a first anchor tip defining a first axis. The first anchor tip is configured to pierce a surface of a tissue wall of the GI tract at a first position and penetrate into the tissue wall in a nonaxial direction relative to the first axis to secure the anchor to the tissue wall. The tether can be cinched to draw the anchors together. Some devices include a locking element to lock the tether after cinching. In some variations of the devices, the anchors are non-plicating. At least one of the plurality of anchors can be self-deforming.

The surface of tissue wall can comprise an interior tissue or an exterior tissue of the GI tract. In some variations of the devices, at least one anchor can penetrate into the muscularis tissue layer. In other variations, at least one anchor can penetrate into the serosa tissue layer.

In some variations of the devices, at least one anchor can comprise a second anchor tip defining a second axis. The second anchor tip can be configured to pierce the surface of the tissue wall at a second position and penetrate into the tissue wall in a nonaxial direction relative to the second axis. In those variations, the at least one anchor can comprise two curved tissue-penetrating legs crossing in a single turning direction. One of the two legs can comprise the first anchor tip and the other of the two legs can comprise the second anchor tip. Each of the legs can form an arcuate shape extending into the tissue wall. The legs can engage the tissue wall in opposing directions that minimize tissue deflection. In some devices, at least one anchor is adapted to gather tissue.

The tether in the devices can comprise any suitable material. In some variations, the tether comprises a suture material. At least one of the plurality of anchors can be slidably coupled to the tether. In other variations, the tether and/or at least one of the anchors can compromise a shape memory material, such as a Nickel-Titanium alloy.

In some devices, the plurality of anchors can comprise a first anchor, a terminal anchor and an intermediate anchor disposed between the first and terminal anchors. In some variations of the devices, the terminal anchor can be fixed to the tether.

Some devices are configured for intraluminal application. For example, devices can be configured for intraluminal application using a delivery device. In those applications, suitable delivery devices include catheters, or steerable catheters. Some delivery devices can be adapted to position the anchors along the surface of the tissue wall and to deploy the anchors to secure the anchors to the tissue wall. Some delivery devices can be adapted to cinch the tether to tighten tissue. Still other delivery devices are configured to lock the tether after the tether has been cinched. In some variations, a delivery device that is configured to deliver and deploy at least two anchors simultaneously can be used to apply the devices described herein intraluminally.

In some devices, at least one of the anchors can comprise a delivery configuration and a deployed configuration. For example, the delivery configuration can be collapsed in at least one dimension and the deployed configuration can be expanded in at least one dimension. For example, the ratio of a diameter of the deployed configuration to a diameter of the delivery configuration can be about 2 to about 20. In some devices having at least one anchor with delivery and deployed configurations, the at least one anchor can comprise two legs. The anchor legs can be compressed in the delivery configuration and expanded in the deployed configuration. Alternatively, the anchor legs can be expanded in the delivery configuration and compressed in the deployed configuration. The at least one anchor can operate to absorb energy during loading of the tissue wall to relieve stress on the tissue wall by collapsing or expanding from its deployed configuration. The anchor legs can be expanded to deploy the anchors into the tissue wall, so that the expansion of the legs drives the anchors into the tissue wall.

Some variations of the devices can include a reinforcing band. Some reinforcing bands can be configured to be joined, e.g., secured, to the tissue wall. Reinforcing bands can be made of any suitable biocompatible material, e.g., DACRON™ polymer.

Methods for restricting a portion of a GI tract are also provided. The methods can be used to treat obesity, GERD, and the like. The methods comprise delivering a plurality of tissue-engageable anchors to a tissue wall of the GI tract. The anchors are coupled to a tether. Each anchor comprises a first anchor tip that defines a first axis. The methods comprise securing each anchor to the tissue wall by piercing a surface of the tissue wall at a first position with the first anchor tip and driving the first anchor tip into the tissue wall in a nonaxial direction relative to the first axis. The methods include cinching the tether to draw the anchors together to tighten the tissue. In the methods described herein, the anchors can be coupled to the tether before or after the anchors are secured to the tissue wall. Some variations of the methods include locking the tether after cinching. The methods can include accessing the GI tract by any suitable method, e.g., laparoscopically or intraluminally.

In some variations of the methods, the anchors can be secured to the tissue wall without plicating the tissue wall. In other variations, at least one anchor is self-deforming and the driving of the first anchor tip of the at least one anchor tip into the tissue wall occurs while the at least one anchor is deforming.

In some variations of the methods, at least one anchor comprises a second anchor tip defining a second axis. The securing of the at least one anchor to the tissue wall comprises piercing the surface of the tissue wall at a second position with the second anchor tip and driving the second anchor tip into the tissue wall in a nonaxial direction relative to the second axis. In these methods, the at least one anchor can comprise two curved tissue-penetrating legs crossing in a single turning direction. One of the two legs can comprise the first anchor tip and the other of the two legs can comprise the second anchor tip. Each of the legs can form an arcuate shape extending into the tissue wall. Some variations of these methods comprise gathering tissue between the two curved legs. For example, the tissue wall can be engaged by the curved legs at spaced apart delivery positions in a delivery configuration. The tissue wall can be gathered together with the legs as the legs approach each other to form a deployed configuration.

The methods can include securing anchors to a tissue wall that comprises interior or exterior tissue of the GI tract. For example, at least one of the anchors can be driven into the muscularis layer of the tissue wall. In other variations of the methods, at least one of the anchors can be driven into the serosa layer of the tissue wall.

In the methods described herein, the tissue wall can comprise at least a portion of a valve. For example, the valve can be the lower esophageal sphincter. In these variations, the methods can be used to treat GERD. The anchors can be positioned circumferentially around the valve and the tether cinched circumferentially to tighten the valve.

In other variations of the methods, the tissue wall can comprise at least a portion of a stomach, so that the cinching of the tether causes restriction of that portion of the stomach. For example, in some variations of the methods described herein, the fundus of the stomach is restricted. The methods can include positioning the anchors circumferentially around a least a portion of the stomach and cinching the tether circumferentially. In other variations of the methods, the stomach can be partitioned into two or more partitions. One of the two or more partitions can comprise at least a portion of the fundus of the stomach.

The methods can also comprise loading at least one anchor into a delivery device. The delivery device can be configured to deliver the at least one anchor to the tissue wall. Further, the delivery device can be configured to deploy the at least one anchor to secure that anchor to the tissue wall. The delivery device in some variations of the methods can hold the anchors in a delivery configuration and deploy the anchors to adopt a deployed configuration. A delivery device can be pre-loaded with at least one anchor. The methods can include inserting and operating the delivery device intraluminally.

Some variations of the methods described herein can include delivering at least two anchors simultaneously to the tissue wall. Further, the methods can comprise deploying at least two anchors simultaneously to secure the at least two anchors to the tissue wall.

Some methods comprise cinching the tether to restrict the portion of the GI tract to a predetermined dimension or a predetermined tension. The tether tension can be adjusted during or after the procedure, e.g., post-operatively. The tension in the tether can be adjusted automatically or manually. In some variations, the methods further comprise reinforcing the tissue wall with a reinforcing band. For example, the reinforcing band can be attached to the tissue wall.

Kits for restricting a portion of a GI tract are also provided herein. The kits provide in packaged combination a plurality of tissue-engageable anchors. Each anchor comprises a first anchor tip defining a first axis. The first anchor tip is capable of piercing a surface of a tissue wall at a first position and penetrating into the tissue wall in a nonaxial direction relative to the first axis to secure the anchor to the wall. The kits also include a cinchable tether for coupling the anchors together. In some kits, the anchors are non-plicating. Kits can include at least one anchor comprising two curved legs crossing in a single turning direction. Some variations of the kits also comprise a delivery device capable of delivering the anchors to the tissue wall. Still other variations of the kits comprise a delivery device capable of securing the anchors to the tissue wall. Kits can comprise a locking element to lock the tether in position and may also comprise a tension-measuring device for gauging tension in the tether, or a tension-setting device for setting tension in the tether. Kits can also include instructions for use.

### BRIEF DESCRIPTION OF THE DRAWINGS

FIGS. 1A and 1B illustrate the human GI tract, and layers of a GI tract tissue wall, respectively.

FIGS. 2A-2F illustrate variations of anchors that can be used in the devices, methods and kits described herein.

FIGS. 3A-3B illustrate a variation of a device and method for restricting tissue in the GI tract.

FIG. 4 shows an expanded partial view of a variation of a device applied to a GI tract via interior access.

FIG. 5 shows an expanded partial view of another variation of a device applied to a GI tract via interior access.

FIG. 6 illustrates a variation of a device in which at least one anchor penetrates into the muscularis layer of the GI tract tissue wall.

FIG. 7 illustrates a variation of a device in which at least one anchor penetrates an exterior surface of a GI tract tissue wall into the serosa layer of the GI tract tissue wall.

FIG. 8 illustrates a variation of an anchor having a single turning direction that can be used in devices, methods and kits described herein.

FIG. 9 illustrates another variation of an anchor having a single turning direction that can be used in devices, methods and kits described herein.

FIG. 10 illustrates a variation of an anchor having a delivery configuration and a deployed configuration that can be used in devices, methods and kits described herein.

FIG. 11 illustrates another variation of an anchor having a delivery configuration and a deployed configuration that can be used in devices, methods and kits described herein.

FIG. 12 illustrates a device having a locking element to lock the tether in position.

FIG. 13 illustrates a variation of a device having a tether comprising a shape memory material.

FIG. 14 illustrates a variation of a device and a method in which the anchor is delivered to the tissue wall with a delivery device.

FIG. 15 illustrates a variation of a device and a method in which a delivery device can be used to deliver multiple anchors to the tissue wall.

FIG. 16 illustrates a variation of a device and method to restrict a portion of a stomach to treat obesity.

FIG. 17 illustrates a variation of a device and method incorporating a reinforcing band.

FIG. 18 illustrates a method for restricting a lower esophageal sphincter to treat GERD.

### DETAILED DESCRIPTION

Described herein are devices, methods and kits for restricting a portion of a GI tract. The devices, methods and kits can be used to treat GERD, obesity, and other disorders of the GI tract that may benefit from the described devices, methods, and kits. The devices, methods, and kits do not require the formation of plications in tissue walls or the formation of tissue mounds. Therefore, the devices, methods and kits described herein may result in faster application, reduced surgical trauma, reduced risk, and/or reduced cost.

The devices comprise a plurality of tissue-engageable anchors coupled to a tether. Each anchor is secured to a tissue wall of the GI tract. The anchors are secured to the tissue wall in a manner that can reduce tissue destruction by axially piercing a surface of the tissue wall with a first anchor tip at a first position, and penetrating that anchor tip into the tissue wall in a nonaxial direction. The tether is configured to be cinched to draw the anchors together, which in turn draws the tissue secured to the anchors together to tighten tissue in a GI tract. The devices, methods and kits can be applied to tissue in any suitable region of a GI tract, e.g., the esophagus to treat GERD, or the stomach to treat obesity.

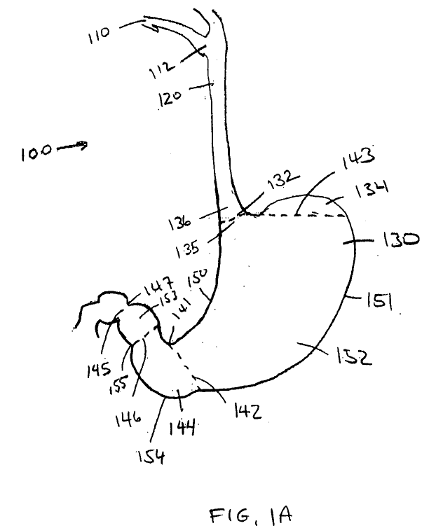

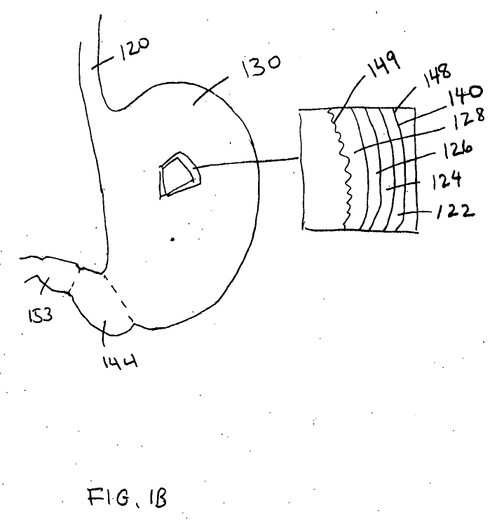

FIGS. 1A-1B illustrate the human GI tract, or alimentary canal. As illustrated in FIG. 1A, food enters the GI tract 100 via mouth (buccal cavity) 110, where the digestive process begins. Food that is swallowed passes through pharynx 112 and enters esophagus 120. The esophagus is a muscular tube that extends from the pharynx 112 to the stomach 130. Food travels down the esophagus and enters the stomach via the lower esophageal sphincter (LES) 132, which is an opening that operates as a unidirectional valve at the junction between the stomach and the esophagus to allow food to pass into the stomach but prevent stomach contents from refluxing back into the esophagus. Thus, the LES should generally be closed when no food is being consumed to prevent bile and stomach acids from refluxing. The presence of food causes muscles in the LES to relax and open, allowing food to enter the stomach.

Referring still to FIG. 1A, stomach 130 includes two openings, two curvatures, and several parts. The two openings are the esophagus and the duodenum. The stomach is open to the esophagus at the cardiac orifice 135. A conical region of the esophagus called the antrum cardiacum 136 adjoins the cardiac orifice. The stomach is open to the duodenum at the pyloric orifice 145. Pyloric orifice 145 is usually defined by a circumferential constricting groove around the stomach, the duodenopyloric constriction 147. The two curvatures are the greater curvature and the lesser curvature. The stomach's lesser curvature 150 forms the right border of the stomach and extends between the cardiac orifice and the pyloric orifice. Near the pyloric orifice, there is an acute angle (incisura angularis) 141 in the lesser curvature. The stomach's greater curvature 151, which is about fives times the length of the lesser curvature, forms the left border of the stomach. On the greater curvature opposite the incisura angularis is an expanded region 154. Expanded region 154 is defined on its right side by a slight circumferential constricting groove 155, known as the sulcus intermedius. The parts of the stomach include the cardia, the fundus, the body, the pyloric part, and the pyloric antrum (pylorus). The cardia is the region of the stomach at the cardiac orifice 135. A horizontal plane 143 passing through the cardiac orifice defines fundus 134 which is an upper or top portion of the stomach. A plane 142 through the incisura angularis divides the stomach into a left stomach part 152, called the body, and a right stomach part 144, called the pyloric part. The pyloric part in turn is divided by a plane 146 passing through the sulcus intermedius 155. Bounded by plane 146 and by pyloric orifice 145 is the pyloric antrum 153. Although a typical adult stomach has a volume of about 1.5 liters, it can be greatly extended, e.g., to a volume of up to 4 liters. Further, the shape of the stomach changes during digestion. For example, during digestion, the left portion of the stomach dilates, whereas the body of the stomach first constricts and then gradually expands as the digestion process continues.

Referring now to FIG. 1B, a GI tract tissue wall has at least four distinct tissue layers and an interior surface and an exterior surface. As shown in the inset, tissue wall 140 has exterior surface 148 and interior surface 149. Still referring to the inset, tissue wall 140 has outer layer 122, which is a serous membrane or serosa. The serosa is a smooth membrane that can excrete serous fluid which acts to lubricate against friction generated by muscle action. Interior to the serosa is a muscle layer or muscularis 124. Interior to the muscle layer 124 is the submucosa 126, which is a layer of loose connective tissue. Finally, the innermost layer of the GI tract is the mucosa 128, which is a layer of epithelium and other loose tissue supported by the submucosa. The mucosa and submucosa are generally fragile layers, and do not have the ability to withstand high tensile loads or other local stresses.

### Devices

In the devices described herein, tissue-engageable anchors are secured to tissue of a GI tract. The anchors are coupled to a tether, and the tether is configured to be cinched to restrict a portion of a GI tract to treat GERD, obesity, and the like. The devices do not require a separate step or procedure to form plications or tissue mounds in the tissue wall to be restricted. That is, the anchors themselves need not plicate tissue, nor must plications or tissue mounds be formed prior to application of the anchors. Although variations of the devices are illustrated herein as being applied via interior access (e.g., intraluminally) to the lumen of a stomach, devices described herein can be applied to other locations or areas of a GI tract (e.g., the esophagus or the pylorus) and can also be applied via exterior access (e.g., laparoscopically).

The anchors of the devices have at least one anchor tip that can penetrate tissue. The anchor tip is a distal section of the anchor that defines an axis just prior to piercing a tissue surface. “Axial” as used herein refers to a direction along the axis defined by a particular tip, whereas “nonaxial” refers to a direction that is not along the axis defined by a particular anchor tip.

Thus, anchors of the devices have a first anchor tip defining a first axis. The first anchor tip axially pierces a surface (i.e., an interior or exterior surface) of a tissue wall of the GI tract at a first position. That is, the first anchor tip pierces the surface in the direction of the first axis. The first anchor tip then penetrates into the tissue wall in a nonaxial direction relative to the first axis to secure the anchor to the tissue wall. The length of the anchor legs may be adjusted so that the legs do not pierce completely through the tissue wall (e.g., perforate a stomach wall or an esophagus wall), which may reduce risks associated with surgical procedures on the GI tract.

In some variations, at least one anchor can have more than one anchor tip that can penetrate tissue. For example, some devices have at least one anchor comprising a second anchor tip defining a second axis. The second tip pierces the surface of the tissue wall at a second position axially, or in the direction of the second axis. The second tip then penetrates into the tissue wall in a nonaxial direction relative to the second axis.

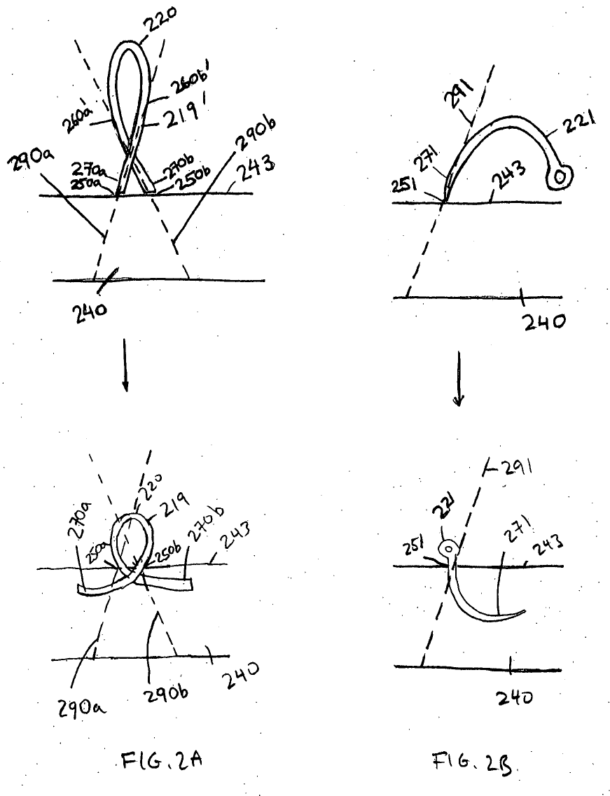

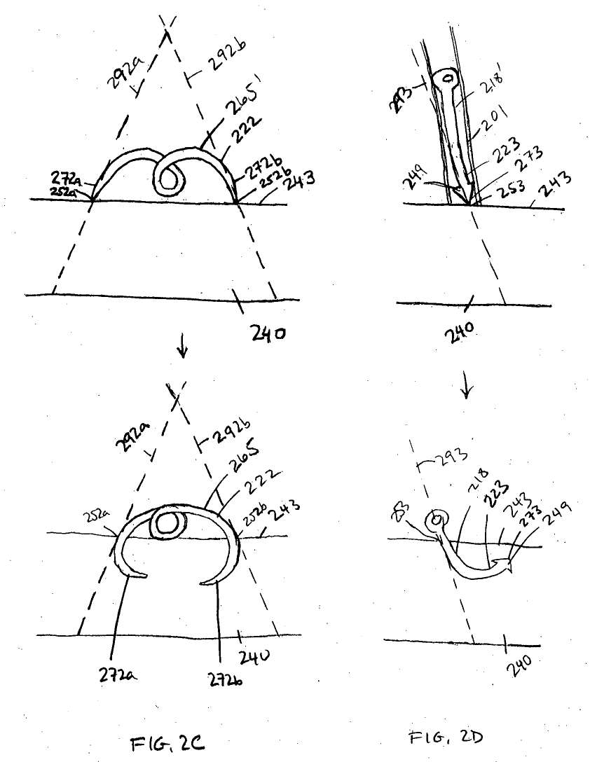

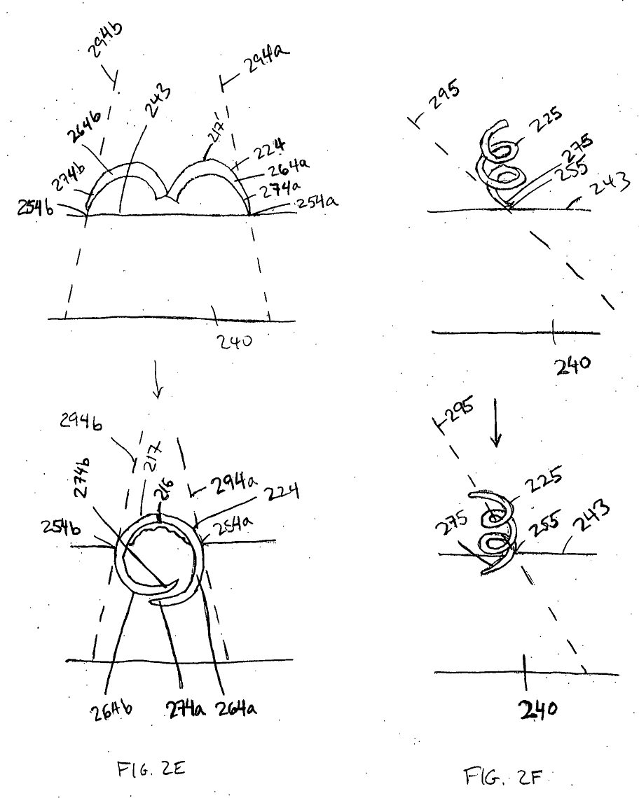

A variety of anchors can be used in the devices described herein. Referring to FIGS. 2A-2F, the anchor tip defines an axis just as it is inserted into tissue, e.g., by a tangent along that tip. Anchor 220 in FIG. 2A has two tips, 270a and 270b. Each tip defines an axis, indicated by dashed lines 290a and 290b. The tips are configured to pierce tissue surface 243 and become secured to and embedded within tissue wall 240. Here, anchor tips 270a, 270b are positioned on surface 243 at positions 250a, 250b, respectively, prior to deployment. Tips 270a, 270b each trace out distinct nonaxial paths that veer away from axes 290a, 290b as they pull anchor 220 into tissue wall 240. Anchor 220 is a self-deforming anchor with a pre-deployment or delivery configuration 219′ and a deployed configuration 219. The anchor legs penetrate into the tissue wall at entrance positions 250a, 250b that are closely spaced together to mirror the close spacing of legs 260a′, 260b′ in delivery configuration 219′. The tissue wall 240 is not generally plicated as the anchor is secured thereto.

Anchor 221 illustrated in FIG. 2B is a curved anchor. Anchor 221 may or may not be self-deforming. Anchor 221 has anchor tip 271 that defines axis 291. Anchor tip 271 pierces surface 243 at position 251 and is driven into tissue wall 240 in a nonaxial direction. FIG. 2C illustrates another variation of a self-deforming anchor. Anchor 222 has anchor tips 272a, 272b defining axes 292a, 292b, respectively, in delivery configuration 265′. Anchor tips 272a, 272b pierce surface 243 at positions 252a, 252b, respectively, and penetrate through tissue wall 240 in nonaxial directions in deployed configuration 265. FIG. 2D illustrates still another variation of a self-deforming anchor. Anchor 223 has tip 273 defining axis 293 as it is positioned to pierce surface 243 at position 253. In this variation, anchor tip 273 comprises a barbed portion 249. Anchor 223 is held in a spring-loaded delivery configuration 218′ by applicator or delivery device 201. Tip 273 traces out a nonaxial penetration path in tissue wall 240 as it uncoils from spring-loaded delivery configuration 218′ to adopt deployed configuration 218.

Anchor 224 as illustrated in FIG. 2E may or may not be self-deforming. Anchor 224 has a first leg 264a with a first tip 274a and a second leg 264b with a second tip 274b. Tips 274a, 274b define axes 294a, 294b, respectively. Legs 264a, 264b may be hinged together (not shown), or they may be separate elements of anchor 223 in its delivery configuration 217′. As tips 274a, 274b pierce surface 243 at positions 254a, 254b, respectively, and penetrate through tissue wall 240, they veer away from axes 294a, 294b, respectively. In this variation, anchor 224 gathers tissue between legs 264a, 264b as it adopts its deployed configuration 217. Anchor 224 may be locked at junction 216 after deployment, e.g., by adhesive, or mechanical locking, or the like. Anchor 225 illustrated in FIG. 2F is helical. Anchor 225 has tip 275 that defines axis 295 as it is prepared to pierce surface 243 at position 255. As helical anchor 225 is twisted into tissue wall 240, tip 275 penetrates along a nonaxial direction relative to axis 295.

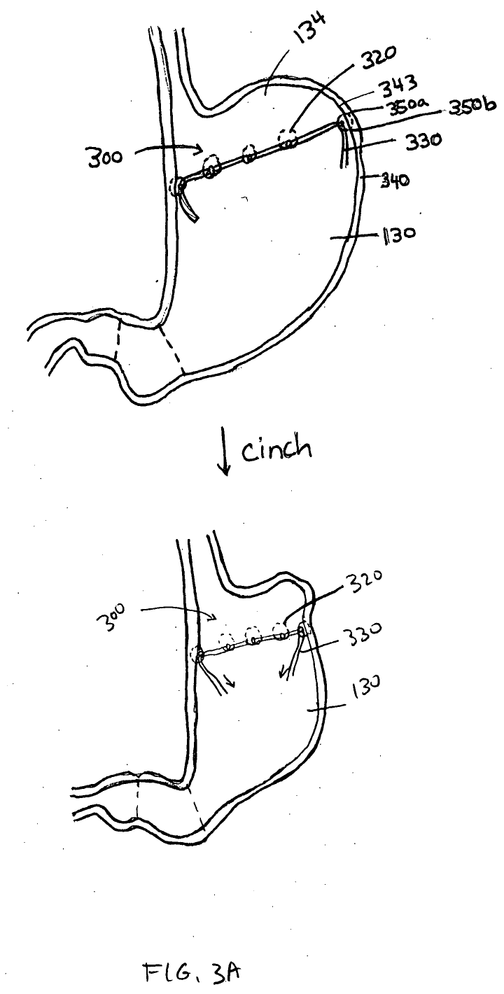

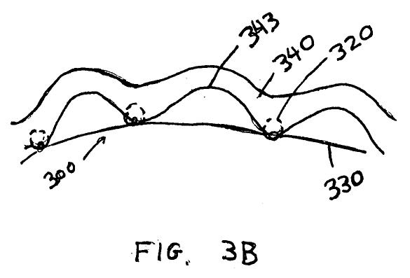

Referring now to FIGS. 3A-3B, device 300 includes anchors 320 coupled to tether 330. In this variation, the anchors 320 are slidably coupled to tether 330. Each anchor 320 is secured to the tissue wall 340 by piercing an interior surface 343 of the tissue wall with a first anchor tip at a first position 350a and a second anchor tip at a second position 350b. Each anchor penetrates nonaxially into tissue wall 340; in this variation the anchors penetrate into the tissue wall between first and second positions 350a and 350a. Tether 330 is configured to be cinched to draw anchors 320 closer together to constrict a portion of the GI tract. In this variation, stomach 130 is partitioned by device 300 and has been substantially reduced in volume. FIG. 3B shows a cross-sectional view of device 300 after tether 330 has been cinched. Tissue wall 340 between anchors 320 can gather as tether 330 is cinched, thereby becoming restricted.

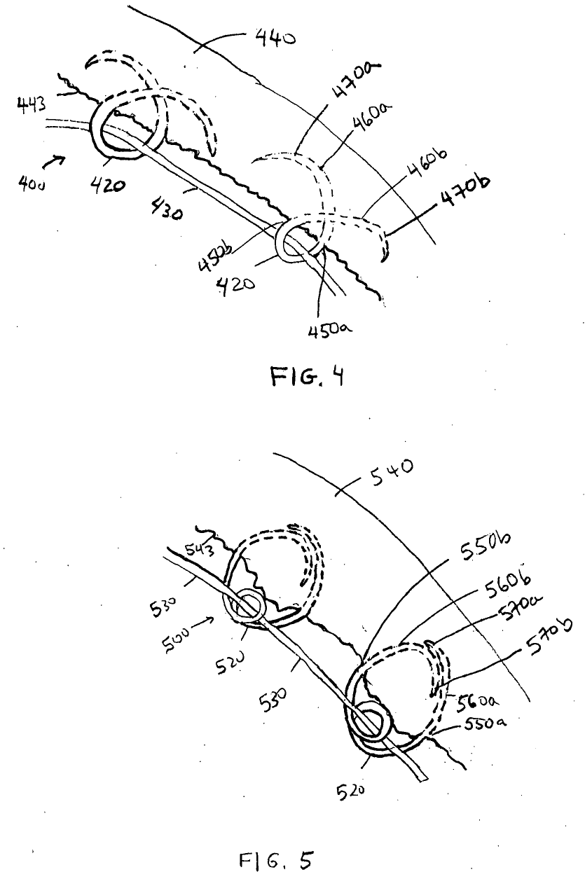

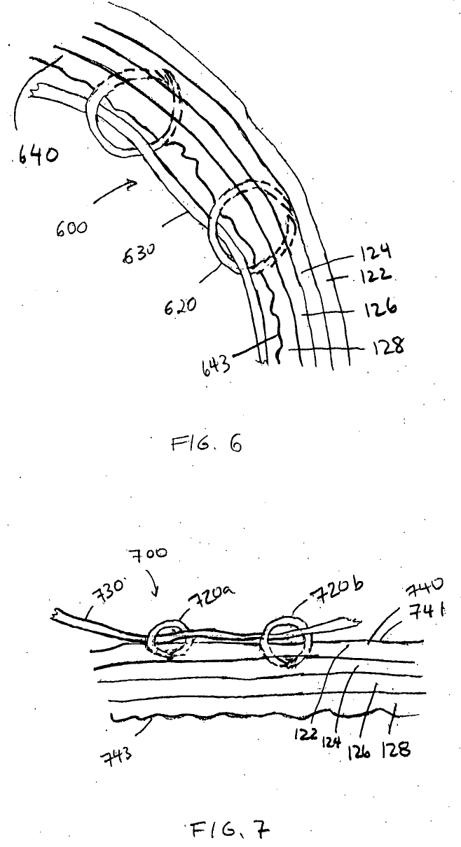

Expanded partial views of devices applied to GI tract via interior access are illustrated in FIGS. 4 and 5. Anchors 420 of device 400 are slidably coupled to tether 430. Anchors 420 have pierced interior surface 443 of the tissue wall 440 at first entrance position 450a with tip 470a of anchor leg 460a and again at second entrance position 450b with tip 470b of anchor leg 460b. In this variation, tips 470a, 470b have penetrated through tissue, away from each other, and away from the original axes defined by the anchor tips. Thus, anchor legs 460a, 460b penetrate into tissue outside of the region between first and second positions 450a, 450b.

As shown in FIG. 5, device 500 has anchors 520 coupled to tether 530. Anchors 520 have pierced interior surface 543 of tissue wall 540 at first entrance position 550a by tip 570a of anchor leg 560a and again at second entrance position 550b by tip 570b of anchor leg 560b. In this variation, anchor tips 570a, 570b have penetrated through tissue toward each other, but away from the original axes defined by the anchor tips. Thus, anchor legs 560a, 560b penetrate into tissue to form a substantially closed configuration in the region between first and second positions 550a, 550b. In this variation, tissue near surface 543 has been gathered between legs 560a, 560b.

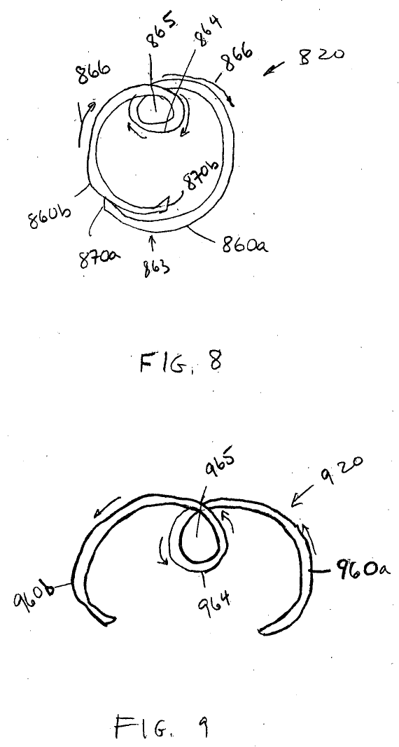

In some variations, as illustrated in FIGS. 6 and 7, at least one anchor in the plurality of anchors of a device can penetrate into the muscularis layer of the issue wall of the GI tract. Referring to device 600 in FIG. 6, anchors 620 are coupled to tether 630. At least one anchor 620 pierces interior surface 643 of the GI tract wall 640 and penetrates through the mucosa 128 and submucosa 126, and into muscularis 124. In other variations (not shown), at least one anchor can penetrate through the mucosa, submucosa and muscularis layers, and penetrate into the serosa layer 122 of the tissue wall. In still other variations, as illustrated in FIG. 7, at least one anchor of a device can penetrate an exterior surface of a tissue wall into the serosa layer of a tissue wall of a GI tract. Device 700 includes anchors 720a, 720b coupled to tether 730. In this variation, device 700 has been applied to the exterior surface 741 of GI tract tissue wall 740. Anchor 720a penetrates only into serosa layer 122, whereas anchor 720b penetrates through serosa layer 122 and into muscularis layer 124.

As stated above, anchors used in the devices can have a variety of configurations and features. For example, the anchors can be flexible. Some anchors can have a delivery configuration that is distinct from a deployed configuration, e.g., the configuration of an anchor prior to being secured into tissue can be distinct from its configuration after it is secured into tissue. Still other anchors can be self-deforming. That is, the anchors can have a deformed state, e.g., prior to deployment, and are capable of recovering to a non-deformed state, e.g., after deployment. In some variations of the devices, the anchors are curved or helical. In still other variations, the anchors can include hinging elements. Some anchors are integral, while other anchors are multi-bodied. Some devices comprise non-plicating anchors, i.e., anchors that do not fold or pleat a tissue wall (that is, they do not gather tissue). However, some anchors applied to an interior or exterior surface of a tissue wall may be plicating and gather a portion of that tissue wall. Some anchors include barbs, spikes, roughened surfaces, or the like, or a combination thereof to enhance their ability to remain embedded in tissue. In many variations, an anchor does not perforate the entire thickness of a tissue wall, i.e., pierce both interior and exterior surfaces. Of course, the devices described herein may include more than one variation of an anchor.

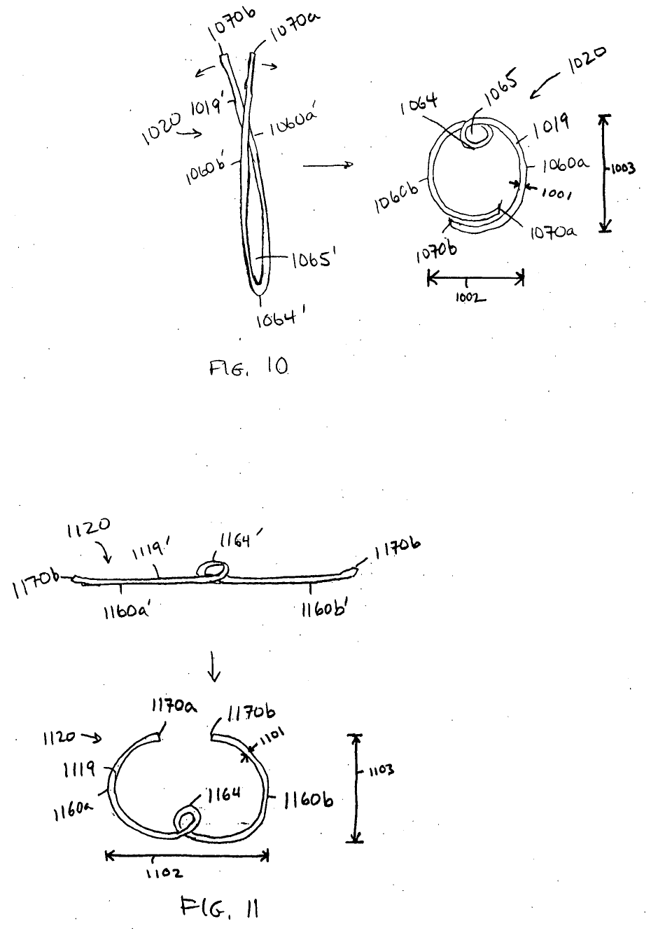

In some variations, the anchors can be flexible anchors having two curved, tissue-penetrating legs. The curved legs can cross in a single turning direction to form a loop. In these variations, the anchor legs can engage the tissue wall in opposing directions to minimize tissue deflection. An example of such a variation of an anchor that can be used in the devices described herein is shown in FIG. 8. Anchor 820 has two curved, tissue-penetrating legs 860a, 860b, and a loop region 865 defined by loop 864. The legs and loop can all have a single turning direction, as indicated by arrows 866. Anchors comprising a single turning direction may be more flexible than anchors having multiple turning directions.

The tissue-penetrating legs of anchors used in the devices described herein can have any type of tip that can penetrate tissue. For example, anchor leg tips can be flat, e.g., end-cut, rounded, pointed, beveled, angled, sharpened, or otherwise adapted to enhance tissue penetration. For example, tips 870a, 870b of legs 860a, 860b, respectively, of anchor 820 in FIG. 8 are pointed and beveled. Further, as illustrated in FIG. 2D, a tip in one or more anchors can be barbed, hooked, and/or roughened to allow for more secure attachment to tissue.

The anchors can have various deployed configurations, i.e., configurations assumed after the anchor is secured to tissue. In some variations, the anchors assume a substantially closed deployed configuration. For example, the anchor illustrated in FIG. 8 is depicted in a deployed configuration. Curved legs 860a, 860b can each form an arcuate shape, e.g., semicircular, on alternate sides of loop region 865. In FIG. 8, legs 860a, 860b are shown as overlapping in region 863 (generally opposite loop region 865) so that the overall deployed shape of anchor 820 is approximately closed. Overlapping legs may either contact each other or overlap without contacting each other. Some anchors may have one leg comprising an orifice into which an opposing leg inserts to form a substantially closed configuration. In other variations, the anchors can assume an open, deployed configuration. For example, as shown in FIG. 9, variations of anchors are contemplated in which the legs do not overlap when deployed. Thus, anchor 920 has two curved, tissue-penetrating legs 960a, 960b formed in a single turning direction on alternate sides of loop region 965 that do not overlap when deployed. The curved, tissue-penetrating legs may have any suitable shape when deployed, e.g., elliptical, uniformly curved, or nonuniformly curved. One or more curved legs may be continuously curved, or may include one or more straight or angled segments in addition to one or more curved segments. In addition, legs in a single anchor can have the same or different shapes, or the same or different lengths.

Anchors may have an eye, eyelet or eye region through which the tether may be threaded. For example, for the anchor variations shown in FIGS. 8 and 9, the loop regions 865, 965, defined by loops 864, 964, respectively, can be referred to as an eye, eyelet or eye region. Although the eyelets in FIGS. 8 and 9 are depicted as single loops, eyelets can comprise multiple loops, e.g., a helical shape having more than one turn.

The anchors in their deployed configuration may be generally planar, meaning that the parts of the anchor including legs occupy approximately the same plane. For example, the anchors illustrated in FIGS. 8 and 9 may be approximately planar. Curved leg 960a can define a plane and curved leg 960b can occupy approximately the same plane. In other variations, the anchors in their deployed configuration can be slightly or substantially non-planar. For example, some anchors may have a non-planar configuration in which the legs define two or more distinct planes. In still other variations, the loop region or eyelet may define a different plane than one or more of the legs. For example, referring to FIG. 8, loop 864 may be in a plane that is out of the plane of the paper, whereas the legs may be substantially in a plane defined by the paper. Of course, variations of anchors are contemplated in which each of the legs and the loop region all occupy distinct planes.

The anchors may include one or more hollow regions. For example, anchors may be formed from a tubular material so that the interior of the anchor is substantially hollow. The hollow interior can be used to house drugs or other healing agents. For example, the interior of one or more anchor legs can be loaded with a healing agent, and the healing agent can be at least partially encapsulated to allow timed or delayed release of the healing agent. In addition, anchors may include one or more holes in the anchor surface. The holes can extend through the thickness of the anchor so that the anchor is at least partially porous. Such holes can act as sites for tissue in-growth to further attach the anchor to tissue.

Anchors may be made of any suitable material, or combination of materials. Suitable materials include biocompatible metals and polymers. For example, anchors can be made of a single wire that is formed into the desired deployed configuration. In other variations, anchors can be formed from a sheet of material, e.g., cut, etched, or stamped. Anchors may be at least partially made of an elastic or superelastic material, e.g., a metal (e.g., spring metal), an alloy, or a polymer (e.g., a rubber, polyethyl ether ketone (PEEK), polyester, nylon, etc.), or some combination thereof that can recover elastically from deformation. For example, anchors can be at least partially made of a shape memory material, e.g., a shape memory metal, such as a Nickel Titanium alloy (e.g., Nitinol), or a shape memory polymer, such as oligo(ε-caprolactone) diol or a polymer or copolymer thereof, or oligo(ε-caprolactone) dimethacrylate or a polymer or copolymer thereof, or oligo(ρ-dioxanone) diol or a polymer or copolymer thereof, etc. In addition to biocompatibility, materials can be chosen for their mechanical characteristics, e.g., strength, stiffness, flexibility, ductility, elasticity, and the like. In some variations, anchors can be made of more than one material. For example, anchor tips and/or legs can be made of a metal that can be sharpened easily, whereas anchor loop region can be made of a material selected to have sufficient stiffness to keep the anchor legs in their deployed configuration without deforming. Anchors may be at least partially biodegradable and/or bioabsorbable. For example, anchors may include a biodegradable coating or be at least partially formed of a biodegradable and/or bioabsorbable material, such as poly(lactic acid), poly(lactic co-glycolic acid), or poly(caprolactone). Thus, these variations of anchors may change in shape, thickness, or other dimension over the time they remain in the body. Further, as anchors change in dimension, e.g., by dissolution or degradation, or by dissolution or degradation of a coating, they may also become more flexible.

As stated above, an anchor can have a delivery configuration that is distinct from its deployed configuration. An anchor's deployed configuration may be a relaxed configuration relative to its delivery configuration, which may be the case for self-deforming anchors. In some variations of the devices, the anchors can absorb energy during loading of the tissue wall to relieve stress on the tissue wall by collapsing or expanding from the deployed configuration. For example, anchors able to absorb energy during loading of a tissue wall may be used in tightening stomach tissue that must continually expand and contract during the digestion process. Further, anchors able to absorb energy during loading of a tissue wall may be used in tightening an LES to relieve stress placed on delicate esophageal tissues. The anchor's delivery configuration can be any configuration in which the anchor is prepared for delivery to the tissue. In some variations, the anchor's legs can be compressed in the delivery configuration so that the anchor has a collapsed profile in at least one dimension. In other variations, the anchor's legs can be expanded in the delivery configuration, again so that the anchor has a collapsed profile in at least one dimension. For example, anchor delivery configurations in which the anchor has an outer diameter (O.D.) of less than about 3.0 mm, or about 2.5 mm, or about 2.0 mm, or about 1.5 mm, or about 1.0 mm, or about 0.5 mm can be used so as to fit into a delivery catheter or delivery device having an inner diameter (I.D.) of about 3.0 mm to about 0.5 mm. In some variations, the anchor may have a delivery configuration having an O.D. of less than about 1.0 mm so that it can be used with a delivery catheter or other delivery device having an I.D. of about 1.0 mm. The ratio of a diameter of a delivery configuration to a diameter of a deployed configuration can be about 1:2 to about 1:20, e.g., about 1:5, or about 1:8, or about 1:10, or about 1:12, or about 1:15, or about 1:18.

FIG. 10 illustrates an anchor having a delivery configuration in which the legs are compressed relative to their arrangement in its deployed configuration. Anchor 1020 has deployed configuration 1019 and delivery configuration 1019′. In delivery configuration 1019′, legs 1060a′, 1060b′ are uncoiled so that they can be extended and aligned approximately longitudinally with respect to each other, with loop region 1065′ enlarged and elongated and anchor leg tips 1070a, 1070b held closely to each other. When delivery configuration 1019′ transforms to deployed configuration 1019, legs 1060a′, 106b′ coil or contract to adapt their curved configurations 1060a, 1060b, resulting in a shortened, less elongated or elliptical loop region 1065. Thus, anchor 1020 can be self-deforming, in the sense that it can automatically revert to its deployed configuration from its delivery configuration. In some variations of the anchors used in the devices described here, the ratio of a diameter of the delivery configuration 1019′ and a diameter of the deployed configuration 1019 can be about 1:2 to about 1:20, e.g., about 1:5, or about 1:8, or about 1:10, or about 1:12, or about 1:15, or about 1:18.

FIG. 11 illustrates an anchor having a delivery configuration in which the legs are expanded relative to its deployed configuration. Anchor 1120 has deployed configuration 1119 and delivery configuration 1119′. In delivery configuration 1119′, legs 1160a′, 1160b′ can be pushed apart in opposite directions so that leg tips 1170a, 1170b are on opposite sides of loop 1164′. When delivery configuration 1119′ transforms to adapt anchor deployed configuration 1119, extended legs 1160a′, 1160b′ coil or contract to adapt their curved configurations 1160a, 1160b around loop 1164. The loop 1164′, 1164 between the anchor legs can act as a spring or hinge to restore the anchor to its deployed configuration, making anchor 1120 self-deforming.

The dimensions of the anchors, including thickness, and deployed width and length, can be adapted for the desired application. For example, the variation of the deployed anchor configuration 1019 illustrated in FIG. 10 has thickness 1001, width 1002, and length 1003, and the deployed anchor configuration 1119 illustrated in FIG. 11 is characterized by thickness 1101, width 1102, and thickness 1103. Anchors used in treating an LES may have smaller dimensions than those used in a stomach to treat obesity. Anchors in their deployed configurations may have a deployed width and/or length of about 1 mm, about 2 mm, about 3 mm, about 4 mm, about 5 mm, about 6 mm, about 7 mm, about 8 mm, about 9 mm, or about 10 mm. In general, the thickness of an anchor may be about 0.10 mm to about 1 mm, e.g., about 0.4 mm, about 0.5 mm, or about 0.75 mm. In some variations, the anchors may have different thicknesses in different regions. For example, an anchor thickness may be different for the loop region than for the legs, e.g., the loop region may be thicker than the legs. Other variations of anchors, in particular self-deforming anchors, can be used in the devices described herein. For example, variations of self-deforming anchors are described in U.S. patent application Ser. No. 11/202,474, filed Aug. 11, 2005, entitled “Devices and Methods for Anchoring Tissue,” which is hereby incorporated by reference in its entirety.

The anchors of the devices can be coupled to the tether in any suitable way. In some devices, anchors can be fixed to the tether. In other devices, as illustrated in FIGS. 3-7, one or more anchors in a device may be slidably coupled to the tether. When an anchor is slidably coupled to a tether, the tether can be threaded through an opening in the body of the anchor, as illustrated for example in FIGS. 6-7, or through an eyelet in the anchor as illustrated in FIGS. 4-5. In some devices, the plurality of anchors can comprise a first anchor, a terminal anchor, and an intermediate anchor disposed between the first and terminal anchor. The terminal anchor can be fixed to the tether, thereby preventing a slidably-coupled anchor from sliding off the tether. Anchors can be fixed to a tether in any suitable manner, e.g., by tying or knotting, using an adhesive, mechanical locking, friction fit, or the like.

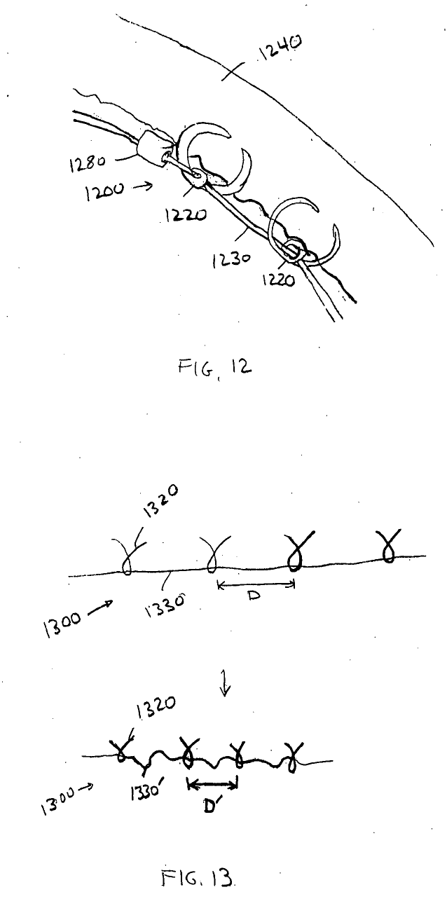

In other devices, one or more locking elements can be applied to the tether to lock the tether in a desired position. The locking element can be applied to both ends of a tether, or can be applied to one end of the tether. The locking element can be any suitable element, e.g., a knot in the tether, a blocking object fixedly coupled to the tether that will not pass through the opening of the anchor through which the tether passes (e.g., an eyelet), a clamp, a crimp, or the like. For example, as illustrated in FIG. 12, device 1200 includes anchors 1220 secured to tissue wall 1240 and slidably coupled to tether 1230, and locking element 1280 fixed to tether 1230. Locking elements can be fixed to a tether in any suitable manner, e.g., by knotting, by using an adhesive, mechanical locking, or using a friction fit, or the like. In some variations, the locking element can be applied in a reversible manner so that the tether can be unlocked, adjusted, and relocked. In other variations, the locking element can be permanently applied.

The cinchable tether of the devices can be made of any suitable biocompatible material. For example, in some variations, the tether can be made of a suture material, e.g., a polymeric or non-polymeric suture. When the tether is made of a suture material, the plurality of anchors can, e.g., be slidably coupled to the tether to allow the tether to be cinched to draw the anchors together. In other variations, the tether can comprise a shape memory material, such as a shape memory metal or alloy, such as a Nickel Titanium alloy (e.g., Nitinol), or a shape memory polymer. Suitable shape memory polymers can include oligo(ε-caprolactone) diol or a polymer or copolymer thereof, oligo(ε-caprolactone) dimethacrylate or a polymer or copolymer thereof, and oligo(ρ-dioxanone) diol or a polymer or copolymer thereof. A tether made of a shape memory material can self-cinch by adopting a constricted state, as illustrated in FIG. 13. Device 1300 includes anchors 1320 coupled to tether 1330. Tether 1330 can be triggered (e.g., by heating to a transition temperature of the shape memory material) to adopt a constricted state 1330′ to cinch the anchors 1320 together to restrict a portion of the GI tract. When a tether is made of a shape memory material, anchors can be fixedly coupled to the tether so that an inter-anchor distance D is contracted to a smaller distance D′, thereby drawing the anchors together and restricting tissue to which the anchors are secured.

Some devices are configured for intraluminal application using a delivery device. The delivery device can be a catheter, e.g., a flexible catheter. In some variations, the delivery device can be a steerable catheter. The delivery device can be adapted to position the anchors along the surface of the tissue wall. Further, the delivery device can be adapted to deploy the anchors to secure the anchors to the tissue wall. In some variations, the delivery device can be configured to deliver and/or deploy anchors in a serial manner, e.g., one at a time. In other variations, the delivery device can be configured to deliver and/or deploy more than one anchor simultaneously. In still other variations, the delivery device can be configured to cinch and/or lock the tether. Some variations of the devices are configured to be applied intraluminally with a delivery device that can be pre-loaded with one or more anchors and/or a tether.

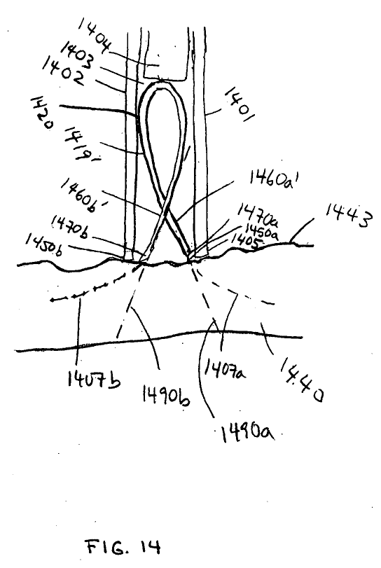

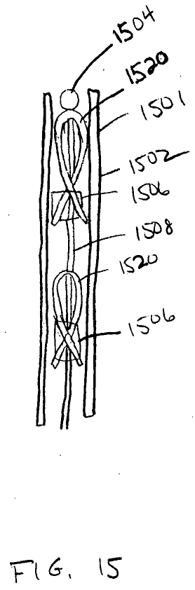

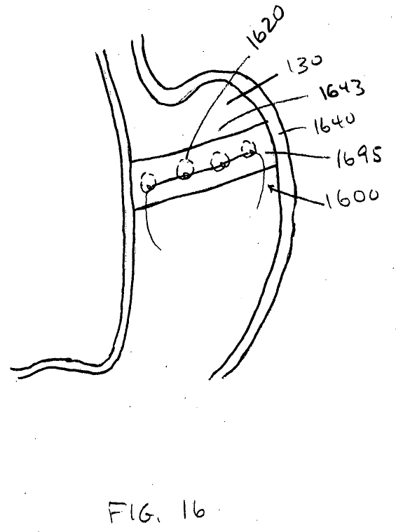

Delivery devices for intraluminal application of the devices described herein can contain a housing for holding one or more anchors, e.g., anchors in a delivery configuration. The housing can have any suitable construction. For example, the housing can be a catheter, a flexible catheter, or a steerable catheter, where one or more anchors are housed in the lumen of the catheter. In addition, delivery devices can include a pusher device configured to push one or more anchors out of the housing. Pusher devices can be operated manually or automatically. As described above, some delivery devices can be pre-loaded with at least one anchor. As illustrated in FIG. 14, delivery device 1401 houses anchor 1420 in a delivery configuration 1419′ similar to delivery configuration 1019′ shown in FIG. 10. The distal end 1405 of delivery device 1401 can urge anchor tips 1470a, 1470b directly against tissue surface 1443. Delivery device 1401 comprises housing 1402, e.g., a catheter, with lumen 1403. Delivery device 1401 can also comprise pusher element 1404 located proximal to anchor 1420. Pusher element 1404 can for example be a rod that can be pushed in a distal direction against anchor 1420 to deploy the anchor into tissue. In other variations, a pull cord (not shown) can be attached to the pusher element, and the pull cord pulled in a distal direction to deploy the anchor into tissue. Anchor tips 1470a, 1470b of legs 1460a′, 1460b′ of delivery configuration 1419′ define axes 1490a, 1490b, respectively. In this variation, anchor tips 1470a, 1470b of delivery configuration 1419′ can penetrate nonaxially into tissue wall 1440 (with trajectories through the tissue wall indicated by dashed lines 1407a, 1407b), thereby pulling the anchor legs into the tissue wall along the same nonaxial paths. The anchor legs penetrate into the tissue wall at entrance positions 1450a, 1450b that are closely spaced together to mirror the close spacing of legs 1460a′, 1460b′ in delivery configuration 1419′. The tissue wall 1440 will not be plicated as the anchor is secured thereto. In addition, anchor legs 1460a′, 1460b′ may be driven deep enough into tissue wall to penetrate into and extend along the muscularis and/or serosa layer in a nonaxial direction from an interior surface. The length of the anchor legs may be adjusted so that the legs will not pierce completely through the tissue wall, which may reduce risks associated with surgical procedures on the GI tract.

Some delivery devices that can be used for intraluminal application of the devices described herein can house more than one anchor. Some of these delivery devices can deliver and/or deploy multiple anchors substantially simultaneously. For example, as illustrated in FIG. 15, delivery device 1501 comprises housing 1502, e.g., a flexible catheter, that houses multiple anchors 1520. Housing 1502 comprises an orifice 1506 corresponding to each anchor. Orifices 1506 can be urged against an interior surface of a GI tract wall. Pusher element 1504 is pulled in a distal direction by applying distal force to pull cord 1508 to force the anchors tip-first out of the orifices and deploy them into tissue. In some variations, a tether (not shown) coupled to anchors 1520 can also be attached to a tether pull cord (not shown). Thus tension can be applied to the tether pull cord to cinch the tether. It should be understood that while FIG. 15 depicts a delivery device in which multiple anchors are delivered and deployed through multiple orifices along the length of the catheter, other variations are possible in which multiple anchors are deployed via the distal end of a single catheter or multiple catheters. The delivery device can be configured to lock a tether in position, e.g., by applying a locking element. Some delivery devices are capable of unlocking and locking a locking element, e.g., during an adjustment of tension on the tether. Other delivery devices include a mandrel holding anchors in a delivery device prior to deployment. For example, anchor legs can be held in a delivery configuration with a mandrel, and legs can be released by the mandrel in a deployed configuration. Examples of such delivery devices are described in U.S. patent application Ser. No. 11/202,474, previously incorporated herein by reference in its entirety.

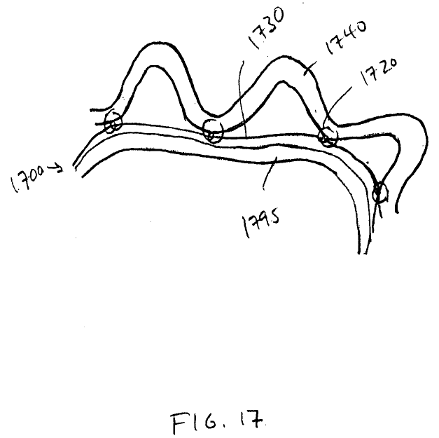

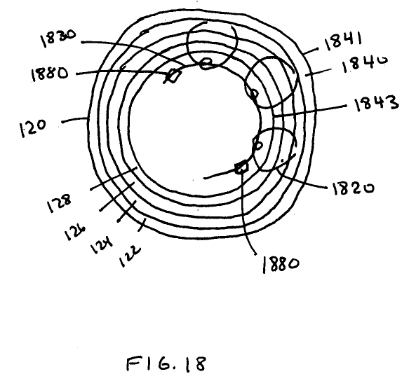

Some variations of the devices comprise a reinforcing band. The reinforcing band can be made from any suitable material, e.g., a biocompatible polymer or mesh. For example, the reinforcing band can comprise a DACRON™ polymer. In other variations, the reinforcing band can be bioabsorbable or biodegradable so that it dissipates over time. In still other variations, the thickness of the reinforcing band can be chosen to reduce an interior dimension of a portion of a GI tract, e.g., stomach. Reinforcing bands can also be selected to have a large surface area so as to reduce the exposed surface area of tissue of a GI tract, e.g., to reduce caloric uptake. In one variation illustrated in FIG. 16, reinforcing band 1695 of device 1600 is overlaid on the surface 1643 of a GI tract wall 1640. In some cases, band 1695 can be secured to surface 1643, e.g., by suturing. Anchors 1620 can pierce through band 1695 to penetrate surface 1643 and reach tissue wall 1640. In another variation illustrated in FIG. 17, reinforcing band 1795 of device 1700 can be overlaid over a section of tissue wall 1740 that has already been restricted by cinching tether 1730 coupled to anchors 1720 secured to tissue wall 1740. Reinforcing bands 1695, 1795 can be secured to a tissue wall, anchors, and/or a tether. For example, reinforcing bands can be secured to a tissue wall using sutures and/or adhesive, and/or secured to anchors or tethers using adhesive and/or any combination of suitable mechanical attachments such as clips, staples, hooks, knots, and the like.

### Methods

Methods are described herein for restricting a portion of a GI tract by tightening tissue. The methods comprise delivering a plurality of tissue-engageable anchors to a tissue wall of the GI tract. The anchors are coupled to a tether. Each anchor comprises a first anchor tip that defines a first axis. The anchors are secured to the tissue wall by axially piercing a surface of a tissue wall at a first position with the first anchor tip and driving the first anchor tip into the tissue wall in a nonaxial direction with respect to the first axis. Thus, in the methods described herein, anchors can be secured to the tissue wall without plicating the tissue wall. The methods include cinching the tether to draw the anchors together, thereby tightening tissue. Several variations of the methods are contemplated to treat obesity, GERD, and the like.

In some variations of the methods, at least one anchor comprises a second anchor tip defining a second axis in addition to the first anchor tip defining the first axis. The at least one anchor can be secured to the tissue wall by piercing the surface of the tissue at a first position with the first anchor tip and driving the first anchor tip into the tissue wall in a nonaxial direction with respect to the first axis, and piercing the surface of the tissue wall at a second position with the second anchor tip and driving the second anchor tip into the tissue wall in a nonaxial direction with respect to the second axis. For example, at least one anchor can comprise two curved tissue-penetrating legs crossing in a single turning direction, with one of the two legs comprising the first anchor tip that pierces the tissue wall at the first position in the direction of the first axis, and the other of the two legs comprising the second anchor tip that pierces the tissue wall at the second position in the direction of the second axis. Each of the two legs can form an arcuate shape extending into the tissue wall. In some variations of the methods, tissue can be gathered between two curved legs. If two curved legs are held at spaced apart delivery positions of a delivery configuration, tissue can be gathered as the legs approach each other to adopt the deployed configuration (see, e.g., FIG. 5).

In some variations of the methods, the anchors can be self-deforming, and the driving of the anchor tip into the tissue wall can occur while the anchors are deforming. FIG. 14 can be used to illustrate a variation of one such method. Anchor 1420 is held in collapsed delivery configuration 1419′ prior to securing into tissue. Tips 1470a, 1470b of collapsed legs 1460a′, 1460b′ abut or urge against interior surface 1443 of tissue wall 1440 at first position 1450a and second position 1450b, respectively. Tip 1470 initially pierces surface 1443 along a direction defined by axis 1490a, and tip 1470b initially pierces surface 1443 along a direction defined by axis 1490b. As collapsed legs 1460a′, 1460b′ adopt their expanded deployed configurations (not shown), tip 1470a bores a path into the tissue wall that is nonaxial with respect to axis 1490a, and tip 1470b bores a path into the tissue wall that is nonaxial with respect to axis 1490b. Anchor legs are thereby drawn into the tissue wall and secured to the tissue wall. By adjusting features of the anchors, e.g., leg length, leg curvature and/or degree of compression in their delivery state relative to that in their deployed state, and by adjusting the force applied during anchor deployment, the depth to which the anchor is embedded into the tissue can be controlled. Thus, the methods can be adapted to restricting different types of tissue within a GI tract. For example, small delicate anchors may be used in restricting a portion of an esophagus, e.g., an LES, whereas larger, more robust anchors may be used in restricting a portion of the stomach.

In the methods described herein, the anchors can be coupled to a tether using any suitable method. Anchors can be slidably coupled to the tether, e.g., through an eyelet, or anchors can be fixed to the tether, e.g., by knotting, using adhesive, friction fit, crimping, clamping, or the like. In some variations of the methods, the tether can be coupled to one or more anchors prior to securing those anchors to the tissue. In other variations, the tether can be coupled to one or more anchors following the securing of that anchor to the tissue, e.g., by threading the tether through a secured anchor. In some variations of the methods, some of the anchors can be attached to the tether prior to securing to tissue and some of the anchors can be attached to tether after being secured to tissue.

Once the anchors are secured to tissue, the methods include cinching the tether to draw the anchors together to restrict or tighten the tissue to which they are secured. Tethers can be cinched in any suitable manner. In some variations, one end of the tether can be fixed and the anchors can be slidably coupled to the tether so that tether can be cinched by pulling on the nonfixed end of the tether. In other variations with slidably coupled anchors, the tether can be cinched by pulling on both ends of the tether. In still other variations, the tether may be self-cinching, e.g., if the tether is made from a shape memory material.

Some variations of the methods include adjusting tension in the tether, either manually or automatically, or by a combination of manual and automatic adjustments. In variations of the methods that include adjustment of tether tension, the tether may be locked into position in a reversible manner, e.g., the methods may include unlocking the tether, adjust the tether and relocking the tether in position. Further, some methods can include measuring the tension in the tether. In some cases, the tether can be accessed and its tension adjusted post-operatively. For example, the tension in a tether can be adjusted post-operatively to accommodate changes in tissue associated with swelling and the healing process, or to tighten or loosen the restriction on the GI tract wall. In some variations, the tether can be cinched to a predetermined tension, while in other variations, the tether can be cinched to a predetermined dimension.

As stated above, the methods allow for restricting a portion of a GI tract without forming plications in the tissue wall with the anchors. In addition, the methods do not require the formation of plications or mounds in the tissue wall prior to securing the anchors thereto. Rather, the methods in some cases result in automatic formation of gathers, plications, or tissue mounds as the tether is cinched. For example, as illustrated in FIG. 3B, cinching of tether 330 has resulted in the formation of tissue mounds between anchors 320. In some variations of the methods, the formation of tissue mounds or tissue plications can be encouraged by manipulating the tissue wall during or after cinching of the tether. Such manipulation may include urging the tissue wall in an exterior or interior direction to determine whether such tissue mounds or plications will be formed toward the interior or exterior of the lumen being restricted.

After the tether is cinched, the methods can include locking the tether to hold a desired tension on the restricted GI tissue. The tether can be locked in any suitable manner. For example, a locking element can be applied to the tether ends. For variations of methods in which the anchors are slidably coupled to the tether, the locking element can be any element that has sufficient diameter so as to not allow the anchor to slide beyond the locking element. The locking element can include one or more knots, clamps, crimps, blocks, or the like. In other variations, the tether itself can be deformed to form a locking element, e.g., by local heating of a tether made from a polymeric or shape memory material. The locking elements can be reversible. For example, the locking element may be a removable clamp that allows subsequent adjustments in tether tension. In some variations, the locking elements can be quasi-permanent or permanent, e.g., a knot or a crimp installed on the tether by a one-way mechanical deformation.

Some methods include loading at least one anchor into a delivery device, wherein the delivery device is configured to deliver the at least one anchor to the tissue wall. One or more anchors can be preloaded in a delivery device, and in some cases, pre-loaded anchors can be coupled to a tether prior to delivery. Some delivery devices are configured to deploy the at least one anchor to secure the at least one anchor to the tissue wall. Methods can also include delivering and/or deploying more than one anchor substantially simultaneously with a delivery device. Still other variations of the methods include cinching the tether with a delivery device. Some methods include locking the tether with the delivery device.

Some variations of the methods are conducted intraluminally. For example, the methods can comprise inserting and operating a delivery device intraluminally. Some examples of variations of delivery devices suitable for use in intraluminal methods are illustrated in FIGS. 14-15. Other variations of the methods can be conducted laparoscopicallly.

The methods described herein can be used to restrict or tighten any suitable part of a GI tract to treat GERD, obesity, or the like. The anchors used in the methods have at least one anchor tip that axially pierces the interior surface of the tissue wall and then penetrates within the tissue wall in a nonaxial direction. The anchor can be embedded into the bulk of the tissue wall that has the greatest physical integrity and therefore the greatest ability to withstand a load, e.g., the muscularis and/or serosa, without tearing or ripping the tissue during the process of embedding. This is because the anchor tip penetrates the tissue in a driving or boring manner, and the body or leg of the anchor can follow the anchor tip into the bulk of the tissue wall.

In some variations of the methods, a reinforcing band can be applied to the tissue to which the anchors will be secured. The reinforcing band can be overlaid on the targeted tissue, and the anchors deployed to penetrate both the reinforcing band and tissue beneath the band. The reinforcing band can be made of any suitable biocompatible material, e.g., a polymer such as a DACRON™ polymer, or a mesh. In some variations, the reinforcing band may be of a biodegradable or bioabsorbable material, e.g., a biodegradable polymer, so that it provides temporary reinforcement during a healing or adjustment stage, and then dissipates. As illustrated by FIG. 16, some variations of the methods can include overlaying a reinforcing band over a surface of the tissue wall before securing the anchors thereto. In still other variations, as illustrated by FIG. 17, some methods can include overlaying a reinforcing band over the anchors and tether after the anchors have been secured to the tissue. The methods can include securing a reinforcing band to a tissue wall, anchors and/or a tether using for example sutures, adhesives, staples, clips, crimps, knots or the like.

Some variations of the methods comprise restricting a portion of a valve or sphincter within a GI tract, e.g., the LES or the pyloric sphincter. GERD can be treated surgically by reinforcing the LES or by restricting the diameter of the esophagus near the LES to limit the backflow of stomach acid. The methods described herein can be used in either approach. For example, anchors can be secured to tissue surrounding the LES intraluminally using the methods described herein. That is, a plurality of flexible anchors coupled to a tether can be secured to the LES tissue wall via the intraluminal access. The tether coupled to the anchor can be cinched so as to draw the anchors together and tighten the tissue around the LES, thereby reinforcing a weak LES that fails to close properly. Esophagus tissue can be fragile, and thus minimized tissue damage will lead to improved methods. In some cases, for treating GERD the anchors can be applied essentially around the entire circumference of the esophagus in the region of the LES. In other cases, anchors can be applied circumferentially around only a portion of the circumference of the esophagus.

Referring now to FIG. 18, in some variations of the methods described herein, the anchors can be secured to interior esophagus tissue circumferential to the region of the LES. Esophagus 120 has tissue wall 1840 with interior surface 1843 and exterior surface 1841. Tissue wall 1840 includes mucosa layer 128, submucosa layer 126, muscularis layer 124 and serosa layer 122. Anchors 1820 are secured to tissue wall 1840 as described above with respect to FIG. 15. That is, anchor tips axially pierce surface 1843 and then bore deep into tissue wall 1840 in a nonaxial manner without piercing exterior surface 1841. As illustrated here, in some variations, the anchors will be embedded in the muscularis layer of the tissue wall. In other variations, the anchors will be embedded in the serosa layer. The anchors, once secured to the tissue wall, can be drawn closer together by cinching tether 1830. Once tether 1830 has been cinched to provide the desired tissue restriction around the LES region of esophagus 120, tether 1830 can be locked in place, e.g., with locking elements 1880. The locking can be reversible to allow for adjustment of tether tension. As discussed above, locking elements 1880 can be any suitable element, e.g., a knot, a crimp, a clamp, a block, or the like. A single device can contain more than one type of locking element, e.g., a permanent locking element on one end and a reversible locking element on the other end. In some variations, a single locking element can be applied to two ends of a tether. The tension in tether 1830 can be adjusted post-operatively in some cases, for example to accommodate changes in tissue associated with swelling and the healing process. Although not illustrated, it is also contemplated that the methods can include securing anchors to exterior tissue of the esophagus in the region of the LES as described above, and cinching the anchors together with a tether to tighten the tissue. Exterior applications of the methods can be conducted by any suitable technique, e.g., using laparoscopic techniques.

The methods described herein can be used to restrict a portion of the stomach to treat obesity. The stomach can be accessed intraluminally, and a series of anchors can be secured circumferentially around the interior of the stomach. The circumferentially-secured anchors are coupled to a tether, and the tether can be cinched to restrict and thereby partition the stomach. By partitioning the stomach such that the upper portion of the stomach has reduced volume, a sensation of fullness can be achieved, causing the patient to consume less food. Further, the reduced volume of the upper part of the stomach may slow down the rate at which food passes into the lower part of the stomach, providing a prolonged sensation of fullness. It is contemplated that the methods described here can be used to restrict a portion of a stomach to treat obesity via intraluminal access, or via exterior access, e.g., using laparoscopy.

For example, as illustrated in FIGS. 3A-3B, a series of anchors 320 can be secured circumferentially around the interior of the stomach wall to partition off a section of stomach 130 including the fundus 134. The anchors can be secured to the stomach wall according to the methods described herein. In some variations, the anchors will be embedded into the muscularis layer of the stomach wall, and in other variations, the anchors will be embedded into the serosa layer of the stomach wall. The anchors are coupled to a tether 330, and tether 330 can be cinched to partition off the upper portion of the stomach as illustrated in FIG. 3A. The tissue wall can be urged outwardly between anchors 330 during or after tightening of tether 330, as illustrated in FIG. 3B. Once a desired amount of restriction has been achieved through the cinching of tether 330, the tether can be locked using locking element (not shown). In some variations, the locking element is reversibly lockable, e.g., a removable clamp or the like, to allow subsequent adjustments of tether 330. Such adjustments may be necessary to accommodate swollen or healing tissue, or to increase or decrease the rate of weight loss experienced by the patient following restriction of his or her stomach. Of course, methods are contemplated in which tissue in other regions of the stomach is tightened, e.g., the cardia, the body, the pyloric part, or the pyloric antrum.

In some variations of methods for treating obesity or GERD, a reinforcing band may be applied circumferentially along an interior stomach surface, for example as illustrated in FIG. 17 or 18. The methods may include selecting a reinforcing band to have a thickness to further restrict an interior diameter of the GI lumen being restricted.

### Kits

Kits are provided for restricting a portion of a GI tract. The kits can be used to treat GERD, obesity, or the like. The kits include in packaged combination a plurality of tissue-engageable anchors, and a cinchable tether. One or more anchors in the kits comprises a first anchor tip defining a first axis. The first anchor tip is capable of piercing a surface of the tissue wall at a first position and penetrating into the tissue wall in a nonaxial direction relative to the first axis to secure the anchor to the tissue wall. In some variations of the kits, the tether can be coupled to the anchors prior to delivery, and in other variations of the kits, the tether is coupled to the anchors after the anchors have been secured to tissue.

The kits can include a delivery device capable of delivering the anchors to the tissue wall. In some kits, the delivery device can be pre-loaded with one or more anchors. In those variations, the delivery device can be pre-loaded with anchors coupled to a tether. In some kits, the delivery device is capable of securing anchors to the tissue and/or cinching the tether. In still other kits, a delivery device can be included that can lock and/or unlock a tether. In some variations of the kits, the anchors are non-plicating. In other variations of the kits, at least one of the anchors comprises two curved tissue-penetrating legs crossing in a single turning direction. Some kits include a locking element for locking the tether after cinching. Some kits include a tension-measuring device for gauging tension in the tether. Other kits include a tension-setting device for setting tension in the tether. Some kits include instructions for use.

What is claimed is:

1. A device for restricting a portion of a gastrointestinal tract comprising:
a plurality of tissue-engageable anchors coupled to a tether, wherein each anchor comprises a first anchor tip defining a first axis, wherein the first anchor tip is configured to pierce a surface of a tissue wall of the gastrointestinal tract at a first position and to penetrate into the tissue wall in a nonaxial direction relative to the first axis to secure the anchor to the tissue wall, and wherein the tether is configured to be cinched to draw the anchors together.

2. The device of claim 1 wherein the anchors are non-plicating.

3. The device of claim 1 wherein at least one of the plurality of anchors is self-deforming.

4. The device of claim 1 wherein at least one anchor of the plurality of anchors comprises a second anchor tip defining a second axis, the second tip configured to pierce the surface of the tissue wall at a second position and to penetrate into the tissue wall in a nonaxial direction relative to the second axis.

5. The device of claim 4 wherein the at least one anchor comprises two curved tissue-penetrating legs crossing in a single turning direction wherein:
one of the two legs comprises the first anchor tip;
the other of the two legs comprises the second anchor tip; and
each of the legs forms an arcuate shape extending into the tissue wall.

6. The device of claim 1 wherein the plurality of anchors comprises a first anchor, a terminal anchor, and an intermediate anchor disposed between the first and terminal anchors.

7. The device of claim 6 wherein the terminal anchor is fixed to the tether.

8. The device of claim 1 wherein at least one anchor is slidably coupled to the tether.

9. The device of claim 1 wherein the tether comprises a suture material.

10. The device of claim 1 wherein the surface of the tissue wall comprises interior tissue of the gastrointestinal tract.

11. The device of claim 1 wherein the surface of the tissue wall comprises exterior tissue of the gastrointestinal tract.

12. The device of claim 1 wherein the first tip of at least one of the plurality of anchors is configured to penetrate into the muscularis tissue layer.

13. The device of claim 1 wherein the first tip of at least one of the plurality of anchors is configured to penetrate into the serosa tissue layer.

14. The device of claim 1 configured for intraluminal application.

15. The device of claim 1 further comprising a locking element to lock the tether after cinching.

16. The device of claim 1 wherein the tether comprises a shape memory material.

17. The device of claim 1 wherein at least one of the plurality of anchors comprises a shape memory material.

18. The device of claim 17 wherein the at least one anchor comprises a Nickel-Titanium alloy.

19. The device of claim 16 wherein the tether comprises a Nickel-Titanium alloy.

20. The device of claim 14 configured for intraluminal application using a delivery device.

21. The device of claim 20 wherein the delivery device comprises a catheter.

22. The device of claim 21 wherein the catheter is steerable.

23. The device of claim 20 wherein the delivery device is adapted to position the anchors along the surface of the tissue wall and deploy the anchors to secure the anchors to the tissue wall.

24. The device of claim 20 wherein the delivery device is adapted to cinch the tether.

25. The device of claim 1 wherein at least one of the plurality of anchors comprises a delivery configuration and a deployed configuration.

26. The device of claim 25 wherein the delivery configuration is collapsed in at least one dimension and the deployed configuration is expanded in at least one dimension.

27. The device of claim 25 wherein the at least one anchor comprises two legs and the legs are compressed in the delivery configuration and the legs are expanded in the deployed configuration.

28. The device of claim 25 wherein the at least one anchor comprises two legs and the legs are expanded in the delivery configuration and the legs are compressed in the deployed configuration.

29. The device of claim 25 wherein the ratio of a diameter of the deployed configuration to a diameter of the delivery configuration is about 2 to about 20.

30. The device of claim 25 wherein the at least one anchor absorbs energy during loading of the tissue wall to relieve stress on the tissue wall by collapsing or expanding from the deployed configuration.

31. The device of claim 5 wherein the legs engage the tissue wall in opposing directions that reduce tissue deflection.

32. The device of claim 27 wherein the legs are expanded to deploy the anchors into the tissue wall so that the expansion of the legs drives the anchors into the tissue wall.

33. The device of claim 20 wherein the delivery device is configured to deliver and deploy at least two anchors simultaneously.

34. The device of claim 20 wherein the delivery device is configured to lock the tether after the tether has been cinched.

35. The device of claim 1 further comprising a reinforcing band.

36. The device of claim 35 wherein the reinforcing band is configured to be joined to the tissue wall.

37. The device of claim 35 wherein the reinforcing band comprises a polymer.

38. The device of claim 1 wherein at least one of the anchors is configured to gather tissue.

39. A method for restricting a portion of a gastrointestinal tract comprising:
delivering a plurality of tissue-engageable anchors to a tissue wall of the gastrointestinal tract, wherein;
the anchors are coupled to a tether; and
each anchor comprises a first anchor tip that defines a first axis;
securing each anchor to the tissue wall by piercing a surface of the tissue wall at first positions with the first anchor tips and driving the first anchor tips into the tissue wall in a nonaxial direction relative to the first axes of the anchors; and
cinching the tether to draw the anchors together.

40. The method of claim 39 wherein the anchors are secured to the tissue wall without plicating the tissue wall.

41. The method of claim 39 wherein at least one of the plurality of anchors is self-deforming, and the driving of the first anchor tip of the at least one anchor into the tissue wall occurs while the anchors are deforming.

42. The method of claim 39 comprising urging the tissue wall between anchors toward the exterior of the gastrointestinal tract.

43. The method of claim 39 wherein at least one of the plurality of anchors comprises a second anchor tip defining a second axis, and the securing of the at least one anchor to the tissue wall comprises piercing the surface of the tissue wall at a second position with the second anchor tip, and driving the second anchor tip into the tissue wall in a nonaxial direction relative to the second axis.

44. The method of claim 43 wherein:
the at least one anchor comprises two curved tissue-penetrating legs crossing in a single turning direction;
one of the two legs comprises the first anchor tip;
the other of the two legs comprises the second anchor tip; and
each of the legs forms an arcuate shape extending into the tissue wall.

45. The method of claim 39 further comprising locking the tether after cinching the tether.

46. The method of claim 39 wherein the anchors are coupled to the tether after the anchors are secured to the tissue wall.

47. The method of claim 39 wherein the anchors are coupled to the tether prior to being secured to the tissue wall.

48. The method of claim 39 comprising loading at least one of the plurality of anchors into a delivery device, wherein the delivery device is configured to deliver the at least one anchor to the tissue wall.

49. The method of claim 48 wherein the delivery device is configured to deploy the at least one anchor to secure the at least one anchor to the tissue wall.

50. The method of claim 39 wherein the tissue wall comprises at least a portion of a valve.

51. The method of claim 50 wherein the valve is the lower esophageal sphincter.

52. The method of claim 50 wherein the anchors are positioned circumferentially around the valve and the tether is cinched circumferentially to tighten the valve.

53. The method of claim 39 for treating gastroesophageal reflux disease.

54. The method of claim 39 for treating obesity.

55. The method of claim 39 wherein the tissue wall comprises at least a portion of a stomach.

56. The method of claim 55 comprising positioning the anchors circumferentially around at least a portion of the stomach.

57. The method of claim 55 comprising restricting the fundus of the stomach.

58. The method of claim 55 comprising partitioning the stomach into two or more partitions.

59. The method of claim 58 wherein one of the two or more partitions comprises at least a portion of the fundus of the stomach.

60. The method of claim 48 comprising inserting and operating the delivery device intraluminally.

61. The method of claim 39 wherein the tissue wall comprises interior tissue of the gastrointestinal tract.

62. The method of claim 39 wherein the tissue wall comprises exterior tissue of the gastrointestinal tract.

63. The method of claim 39 comprising driving the first anchor tip of at least one of the plurality of anchors into the muscularis tissue layer of the tissue wall.

64. The method of claim 39 comprising driving the first anchor tip of at least one of the plurality of anchors into the serosa tissue layer of the tissue wall.

65. The method of claim 39 comprising accessing the gastrointestinal tract laparoscopically.

66. The method of claim 39 comprising accessing the gastrointestinal tract intraluminally.

67. The method of claim 49 wherein the delivery device holds the anchors in a delivery configuration and deploys the at least one anchor to adopt a deployed configuration.

68. The method of claim 39 comprising delivering at least two anchors simultaneously to the tissue wall.

69. The method of claim 39 comprising deploying at least two anchors simultaneously to secure the at least two anchors to the tissue wall.

70. The method of claim 39 comprising cinching the tether to a predetermined tension.

71. The method of claim 39 comprising cinching the tether to a predetermined dimension.

72. The method of claim 39 further comprising adjusting tension in the tether.

73. The method of claim 72 wherein the tension is adjusted automatically.

74. The method of claim 72 wherein the tension is adjusted manually.

75. The method of claim 39 comprising reinforcing the tissue wall with a reinforcing band.

76. The method of claim 44 comprising gathering tissue between the two curved legs.

77. The method of claim 76 comprising engaging the tissue wall with the legs at spaced apart delivery positions in a delivery configuration and gathering the tissue wall together as the legs approach each other to form a deployed configuration.

78. A kit for restricting a portion of a gastrointestinal tract comprising in packaged combination:
a plurality of tissue-engageable anchors, each anchor comprising a first anchor tip defining a first axis, wherein the first anchor tip is capable of piercing a surface of a tissue wall at a first position and penetrating into the tissue wall in a nonaxial direction relative to the first axis to secure the anchor to the tissue wall;
a cinchable tether for coupling the anchors together; and
a delivery device capable of delivering the anchors to the tissue wall.

79. The kit of claim 78 wherein the delivery device is capable of securing the anchors to the tissue wall.

80. The kit of claim 78 wherein the anchors are non-plicating.

81. The kit of claim 78 comprising a tension-measuring device for gauging tension in the tether.

82. The kit of claim 78 comprising a tension-setting device for setting tension in the tether.

83. The kit of claim 78 further comprising instructions for use.
